(19) United States
(12) Patent Application Publication
(10) Pub. No.: US 2018/0193543 A1
(43) Pub. Date: Jul. 12, 2018

(54) IMPLANTABLE INTRAVASCULAR VENTRICULAR ASSIST DEVICE

(71) Applicant: Benjamin Sun, Eden Prairie, MN (US)

(72) Inventor: Benjamin Sun, Eden Prairie, MN (US)

(21) Appl. No.: 15/401,189

(22) Filed: Jan. 9, 2017

(51) Int. Cl. A61M 1/12; A61N 1/39; A61M 1/10; A61F 2/24

(52) U.S. Cl. CPC A61M 1/125; A61M 1/127; A61F 2/2421; A61M 1/1036; A61M 1/122; A61N 1/3962

(57) ABSTRACT

The present disclosure provides intravascular ventricular assist devices for insertion into the vasculature of an individual to improve the overall blood flow of the individual. In many embodiments, the intravascular ventricular assist devices described herein provide an individual with an intravascular ventricular assist device that is sized and configured for insertion into an aortic root or pulmonary root such that its reduced size and placement provide the individual with an improved quality of life. The intravascular ventricular assist devices described herein utilize a pump and optionally a prosthetic valve in combination with a self-expandable or balloon expandable stent or frame to allow placement within the aortic root or the pulmonary root such that a functioning valve works in combination with the pump to increase blood flow within the heart. Both intravascular left ventricular assist devices and intravascular right ventricular assist devices are within the scope of the present disclosure. Processes for implanting the intravascular ventricular assist devices are also disclosed herein.

### BACKGROUND OF THE DISCLOSURE

### a. Field of the Disclosure

The present disclosure generally relates to implantable intravascular ventricular assist devices and methods of making, using, and implanting the same for addressing congestive cardiac failure and other heart conditions. In particular, the present disclosure relates to an implantable intravascular ventricular assist device including an expandable frame sized and configured for insertion into the aortic root or the pulmonary root. The device generally includes a prosthetic valve disposed in the expandable frame and a pump secured to the expandable frame for improving the overall blood circulation of an individual.

### b. Background Art

Congestive heart failure (CHF), also referred to as heart failure or congestive cardiac failure (CCF), is the pathophysiologic state in which the heart, via an abnormality of cardiac function, fails to pump blood at a rate commensurate with the requirements of the metabolizing tissues. Heart failure may be caused by cardiomyopathy, heart valve damage, coronary heart disease, hypertension, and in some cases, diabetes. The heart compensates for the pumping insufficiency by dilating the ventricular chambers, thickening the walls (hypertrophy), and accelerating the pulsation rates.

More than 11 million patients currently suffer from CHF worldwide with an estimated annual increase of about ten percent of cases. Approximately one million individuals present severe CHF conditions, and one percent are admitted in terminal condition. CHF is generally considered the fastest-growing clinical cardiac disease entity in the United States, affecting nearly two percent of the population. Nearly one million hospital admissions for acute decompensated CHF occur in the United States yearly, almost double the number seen 15 years ago. The re-hospitalization rates during the six months following discharge are as high as fifty percent. Nearly two percent of all hospital admissions in the United States are for decompensated CHF, and heart failure is the most frequent cause of hospitalization in patients older than 65 years. Despite many aggressive therapies, hospital admissions for CHF continue to increase.

Heart transplants have been the highest standard of treatment for end-stage CHF. A heart transplant is the replacement of a diseased heart with a healthy one from an organ donor. Candidates for transplant have irreparably damaged hearts, are facing imminent death, and have otherwise viable vital organs. Transplanted hearts generally fail about ten years on average after implantation. Most patients spend months or years waiting for a suitable donor heart and many die before one becomes available.

A ventricular assist device (VAD) is an electro-mechanical device that is used to partially or completely replace the function of a failing heart. Some VADs are intended for short term use, typically for patients recovering from heart attacks or heart surgery, while others are intended for long term use (months to years and in some cases for life), typically for patients suffering from CHF. VADs are distinguished from artificial hearts, which are designed to completely take over cardiac function and generally require the removal of the patient's heart. VADs are generally designed to assist either the right (RVAD) or left (LVAD) ventricle, although a RVAD and an LVAD can be used at the same time (biventricular assist device). The choice of device depends on the underlying heart disease and the pulmonary arterial resistance which determines the load on the right ventricle. LVADs are most commonly used but when pulmonary arterial resistance is high, right ventricular assist becomes necessary. Long term VADs are normally used to keep patients alive with a good quality of life while they wait for a heart transplant.

Most VADs operate on similar principles. A cannula is inserted into the apex of the appropriate ventricle. Blood passes through this into a pump and then through a graft attached to the aorta in the case of an LVAD or to the pulmonary artery in the case of an RVAD. The pump is powered through a lead which connects it to a controller and power supply. Some VADs emulate the heart by using a pulsatile action where blood is alternately sucked into a blood chamber within the pump from the left ventricle then forced out into the aorta. These are volume displacement pumps. Other VADs are based on intravascular continuous flow pumps, which can be roughly categorized as either centrifugal pumps or axial flow impeller driven pumps. These VADs have impellers with high flow rate capability and are much smaller than traditional VADs.

### SUMMARY OF THE DISCLOSURE

The present disclosure is generally directed to left ventricular assist devices and right ventricular assist devices implanted into the vasculature of an individual to improve the overall blood flow of the individual. In many embodiments, the ventricular assist devices described herein provide an individual with a device that is sized and configured for insertion and securement into the aortic root or pulmonary root such that its reduced size and intravascular placement provide the individual with an improved method of deployment and improved blood flow. In many embodiments, the pumping function of another ventricular assist device may be maintained. In some embodiments, the device is sized and configured for insertion and securement into the native aortic valve or native pulmonary valve such that the native valve is held continuously in an open conformation. The ventricular assist devices described herein may be designed and configured to work both with fully functioning native valves and without fully functioning native valves. The devices generally utilize a pump and a prosthetic valve in combination with a self-expandable or balloon expandable stent or frame to allow placement and securement within the aortic root or pulmonary root such that a functioning valve works in combination with the pump to increase blood flow within the heart. The pump is desirably secured to the expandable frame to prevent dislodgement therefrom. Both left and right ventricular assist devices are within the scope of the present disclosure. Processes for implanting the ventricular assist devices are also disclosed herein.

The present disclosure is directed to an intravascular ventricular assist device. The intravascular ventricular assist device comprises: (i) an expandable frame sized and configured for insertion into an aortic root and includes a receiving means thereon; and (ii) a pump disposed within the expandable frame and including attachment means configured to align with the receiving means disposed on the expandable frame to allow securement of the pump to the expandable frame.

The present disclosure is further directed to a process for implanting an intravascular ventricular assist device. The process comprises: (i) inserting a guidewire through a native aortic valve; (ii) delivering over the guidewire an expandable frame comprising a valve positioned therein into an aortic root; (iii) deploying the expandable frame comprising the valve positioned therein; and (iv) delivering a pump into the expandable frame.

The present disclosure is further directed to a process for implanting an intravascular ventricular assist device. The process comprises: (i) inserting a guidewire through a native aortic valve; and (ii) delivering over the guidewire an intravascular ventricular assist device, wherein the intravascular ventricular assist device comprises: (a) an expandable frame sized and configured for insertion into an aortic root; (b) a valve disposed in the expandable frame; and (c) a pump disposed within the expandable frame and including attachment means configured to align with receiving means disposed on the expandable frame to allow securement of the pump to the expandable frame.

The present disclosure is further directed to an intravascular ventricular assist device. The intravascular assist device comprises: (i) an expandable frame sized and configured for insertion into a pulmonary root and comprises a receiving means thereon; (ii) a valve disposed in the expandable frame; and (iii) a pump disposed within the expandable frame and including attachment means configured to align with the receiving means disposed on the expandable frame to allow securement of the pump to the expandable frame.

The foregoing and other aspects, features, details, utilities, and advantages of the present disclosure will be apparent from reading the following description and claims, and from reviewing the accompanying drawings.

### BRIEF DESCRIPTION OF THE DRAWINGS

FIG. 1 shows a perspective view of an expandable frame having a prosthetic valve disposed therein.

FIG. 2 shows a top down view of the expandable frame and prosthetic valve of FIG. 1 wherein the valve leaflets are in a closed conformation.

FIG. 3 shows a top down view of the expandable frame and prosthetic valve of FIG. 1 wherein the valve leaflets are in an open conformation.

FIG. 4 shows a perspective view of an expandable frame having a prosthetic valve disposed therein wherein the frame/valve is disposed in a native aortic valve opening.

FIG. 5 shows a pump including multiple attachment fins thereon.

FIG. 6 shows the expandable frame and prosthetic valve of FIG. 1 further including a rail system on the expandable frame.

FIG. 7 shows a perspective view of an expandable frame including a prosthetic valve and pump disposed therein.

FIG. 8 shows a top down view of an expandable frame including a prosthetic valve and pump disposed therein wherein the valve and pump are coaxially aligned.

FIG. 8A shows a perspective view of an expandable frame and a pump disposed therein in accordance with one embodiment of the present disclosure.

FIGS. 9A, 9B, 10A, and 10B are flow charts of two embodiments for processes for implanting an intravascular ventricular assist device of the present disclosure.

### DETAILED DESCRIPTION OF THE DISCLOSURE

The present disclosure is generally directed to intravascular ventricular assist devices that are sized and configured for insertion and securement into an aortic root or sized and configured for insertion and securement into a pulmonary root. The intravascular ventricular assist devices described herein may be suitable for both short-term and long-term use by a patient, and are suitable for both adult and pediatric patients. These devices generally include an expandable frame or cage that has disposed therein a prosthetic valve and a pump, although expandable frames including a pump only are also within the scope of the present disclosure. The pump is connected to a controller/power supply unit and optionally to one or more peripherals, such as an implantable cardioverter defibrillator or the like, to allow the pump, controller, and peripheral(s) to effectively communicate. The intravascular ventricular assist devices as disclosed herein provide a number of unique advantages to an individual in addition to improving the overall blood flow of the individual. For example, the devices disclosed herein, in many embodiments, are substantially more compact than traditional implantable ventricular assist devices; in addition, the insertion, deployment, and securement of this device may be performed without a full sternotomy through minimal access approaches, and, as such, may be suitable for use in a greater number of individuals to further improve the overall quality of life of the individuals. Further, in some embodiments, the intravascular ventricular assist device, controller/power supply, and any peripherals may all be contained within the body, thus negating the need to have any electrical or other components extend from the interior of the body to the exterior.

Although the present disclosure is primarily described in relation to left intravascular ventricular assist devices that are sized and configured for insertion and securement into an aortic root and fixated to the aortic annulus, as noted above right intravascular ventricular assist devices for use in the pulmonary root and processes and methods related thereto are also with the general scope of the present disclosure.

As noted, the intravascular ventricular assist devices described herein allow for the temporary or permanent implantation of the device inside of the aortic or pulmonary root such that the need to open and access the chest of the patient is minimized or eliminated. The intravascular ventricular assist devices described herein may be implanted into an individual using a variety of processes as further described herein. In some embodiments, the device may be implanted into a patient in a single implantation step. In this embodiment, the expandable frame, prosthetic valve, and pump are preassembled and introduced into the individual, using a guidewire for example, as a single unit. In other embodiments, the device may be implanted into an individual in two steps. This embodiment implants and deploys the expandable frame/prosthetic valve first followed by introducing and securing the pump within the expandable frame. This approach may allow for a lower profile delivery system. The implantation/deployment processes described herein allow for the operator to receive both visual and tactile information to ensure proper placement/deployment of the intravascular ventricular assist device.

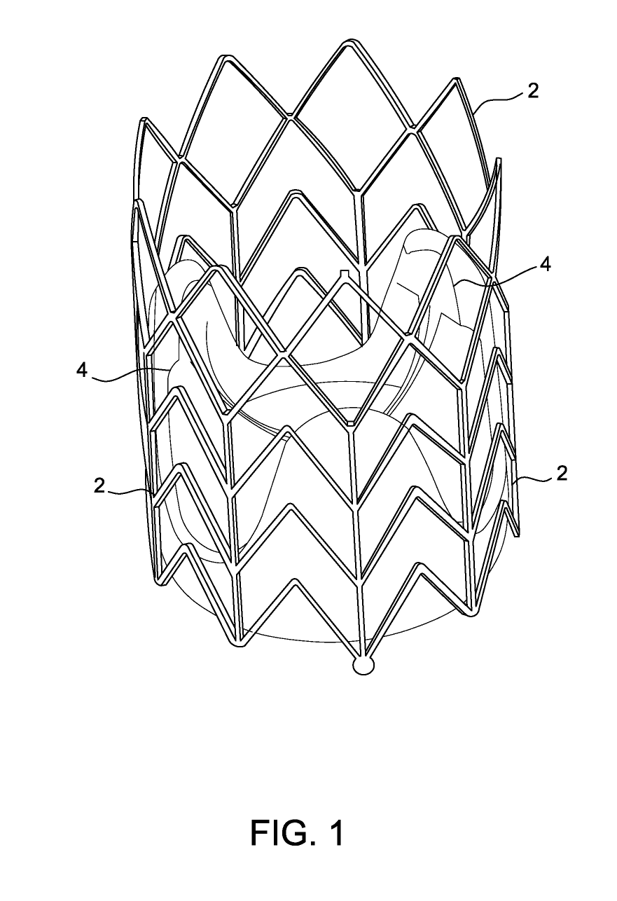

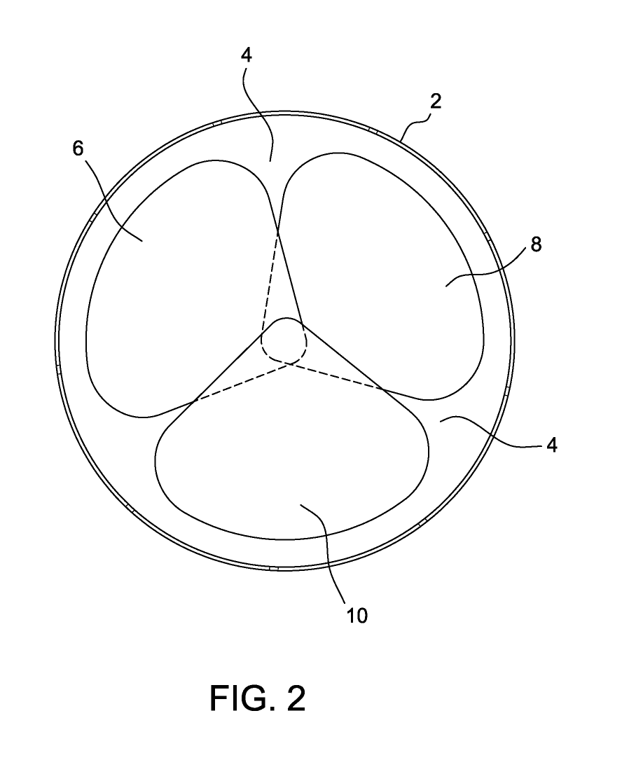

As noted above, the intravascular ventricular assist devices of the present disclosure generally include an expandable frame or cage (also referred to as a “stent”) and a prosthetic valve disposed therein. Although the prosthetic valve is not required in all embodiments described herein as noted above, the present disclosure and Figures are described primarily using a combination expandable cage and prosthetic valve. Referring now to the Figures, and specifically to FIG. 1, there is shown an expandable frame 2 having disposed therein prosthetic valve 4. Prosthetic valve 4 is affixed to expandable frame 2 using methods known in the art such that prosthetic valve 4 is secured to expandable frame 2 for use in the human body. Expandable frame 2 may be comprised of nitinol, stainless steel, or another shape memory metal, material, or alloy, generally formed into a tubular mesh or mesh-like material. The exact construction material of expandable frame 2 is not critical, so long at the beneficial shape memory characteristics and desired strength are provided.

Expandable frame 2 may be a self-expanding frame such that upon the removal of a force (a delivery catheter or delivery sheath, for example) the self-expanding frame expands from its collapsed state to its full predetermined shape and retains that shape indefinitely. Alternatively, expandable frame 2 may be a balloon-expandable frame that may be, for example, constructed from stainless steel wire secured on an inflatable balloon. Other types of expandable frames are also within the scope of the present disclosure for use as expandable frame 2.

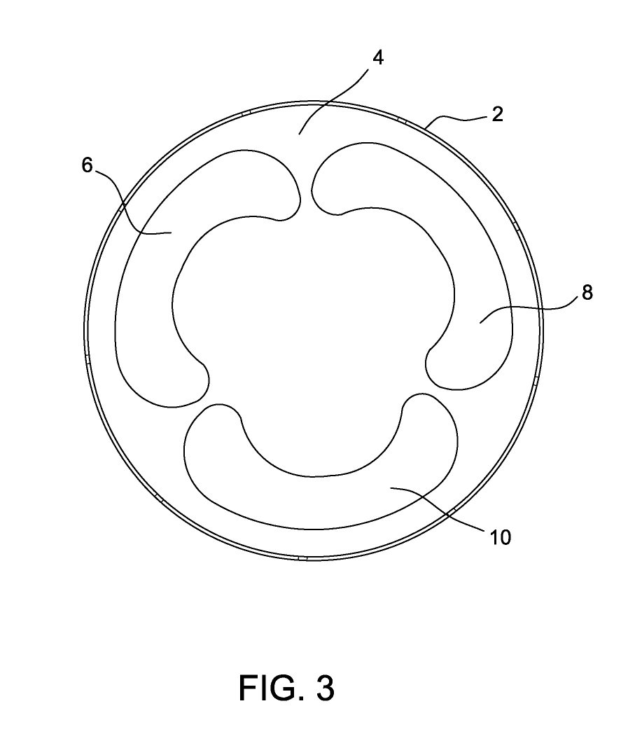

Prosthetic valve 4 disposed in expandable frame 2 may be any suitable prosthetic heart valve including, for example, mechanical prosthetic heart valves (bileaflet, single tilting disk, caged ball, and the like) and biologic or tissue-based prosthetic heart valves. Referring now to FIG. 2, there is shown a top down view of expandable frame 2 and prosthetic valve 4 of FIG. 1 wherein prosthetic valve 4 includes valve leaflets 6, 8, and 10. Valve leaflets 6, 8, and 10 in FIG. 2 are shown in closed conformation as would be expected when the heart is at rest, wherein a portion of each of valve leaflets 6, 8, and 10 overlap one another to form a seal or closure. Referring to FIG. 3, valve leaflets 6, 8, and 10 of prosthetic valve 4 disposed in expandable frame 2 are shown in an open conformation, or the conformation that would be expected when the heart is pumping blood such that the blood may pass through prosthetic valve 4. Although illustrated in FIGS. 2 and 3 as a prosthetic valve having three leaflets, other prosthetic valve types as described above may also be used in accordance with the present disclosure. In many embodiments, prosthetic valves having two or three leaflets are desirable such that they may easily close around a pump, as described below.

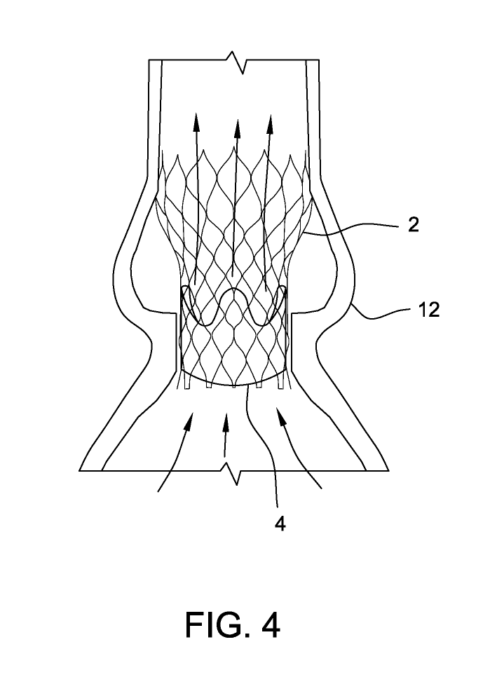

As noted above, the intravascular ventricular assist devices of the present disclosure in many embodiments are sized and configured for insertion and securement into an aortic root or specifically into a native aortic valve to allow for improved blood flow in an individual. Referring now to FIG. 4, there is shown expandable frame 2 including prosthetic valve 4 positioned with native aortic valve 12 such that blood (not shown in FIG. 4) can flow through prosthetic valve 4 along the path of the arrows and into the ascending aorta (not shown in FIG. 4). Although the native aortic valve is held open continually (and thus is non-functioning) after expandable frame 2 including prosthetic valve 4 is introduced therein and deployed as discussed hereinbelow, blood can still pass through prosthetic valve 4 and into the ascending aorta when the heart pumps.

The intravascular ventricular assist devices of the present disclosure include a pump located and secured in the expandable frame (and within the prosthetic valve when present) described above to pump blood and improve the overall blood flow of an individual. The pump may be sized and configured for insertion into a prosthetic valve disposed in the expandable frame such that the prosthetic valve can open and close and perform its intended function without impact from pump. The exact type, size, shape, configuration, and construction of the pump is not critical, so long as the pump may be sized and configured for insertion and securement into the expandable frame/prosthetic valve and has the desired pumping rate of at least about 3 L/min, or even about 4 L/min, or even about 5 L/min, or even about 6 L/min, or even about 7 L/min, or even about 8 L/min. The pump may include one or more drivelines to allow for electrical connection with one or more peripherals such as a power supply, controller, another cardiac device, etc. The one or more drivelines may be routed in/out of the patient at the same access site used to implant and deploy the device. Alternatively, the pump may be powered wirelessly through transcutaneous energy transfer or the like and may also communicate with a controller unit and other peripherals wirelessly. When a driveline is utilized to power and control the pump, it is generally desirable to route the driveline through the expandable frame in such a manner that is does not interfere with the workings of the prosthetic valve. For example, in one embodiment, the driveline may be routed along the expandable frame such that it does not interfere with the prosthetic valve.

Although not required in every embodiment, in many embodiments, it is desirable for the pump to be axially aligned with the prosthetic valve within the expandable frame to further the flow of blood through the prosthetic valve. Generally, the pump and the prosthetic valve are axially aligned and positioned along a major longitudinal axis of the expandable frame to further the flow of blood through the prosthetic valve and minimize or eliminate pump interference with the operation of the prosthetic valve. Alternatively, a centrifugal pump may be used.

Pumps suitable for use in the intravascular ventricular assist devices of the present disclosure can be divided into two main categories; (i) pulsatile pumps that mimic the natural pulsing action of the heart, and (ii) continuous flow pumps. Pulsatile-type intravascular ventricular assist devices use positive displacement pumps. In some of these pumps, the volume occupied by blood may vary during the pumping cycle.

Continuous flow-type pumps generally use either a centrifugal pump or an axial flow pump. Both types have a central rotor containing permanent magnets. Controlled electric currents running through coils contained in the pump housing apply forces to the magnets, which in turn cause the rotors to spin. In the centrifugal pumps, the rotors are shaped to accelerate the blood circumferentially and thereby cause it to move toward the outer rim of the pump, whereas in the axial flow pumps the rotors are more or less cylindrical with blades that are helical, causing the blood to be accelerated in the direction of the rotor's axis. With continuous flow pumps the method used to suspend the rotor may include the use of solid bearings, the use of magnetic levitation (“maglev”) or the use of hydrodynamic suspension. These types of pumps may be desirable as they contain only one moving part (the rotor). In some embodiments of the present disclosure, a suitable pump for use may include a hemo pump, which is a continuous flow pump that is not synced to the rhythm of the heart.

As noted herein, the pump contained in the prosthetic valve disposed in the expandable frame is desirably secured to the expandable frame for use inside of the aortic root. This securement is desirable as the pumping action of a beating heart will tend to displace the pump from the expandable frame if the pump is not secured thereto. By securing the pump to the expandable frame, the pump may be consistently held in the desired position during use (generally axially aligned with the prosthetic valve along a major longitudinal axis of the expandable frame). The pump may be designed and configured in any suitable manner to allow for securement to the expandable frame while not significantly impacting the function of the prosthetic valve, if present. Generally, the pump will include one or more attachment means for securing the pump to the expandable frame at one or more positions on the expandable frame. In one embodiment, the pump may include an attachment means designed to engage a rail system or similar securement system that is installed on the expandable frame to provide the desired securement. In this embodiment, the pump may include one or more securement posts or fins sized and configured to engage one or more guide rails on the expandable frame. Although described primarily herein with respect to the use of securement posts or securement fins located on the motor to attach and secure the motor to the expandable frame, the motor may be sized and configured to include any suitable means for allowing the desired attachment and securement to the expandable frame. For example, in some embodiments, the motor may be sized and configured to include hooks, barbs, and/or tethers/wires thereon that may be suitable for attachment to the expandable cage at one or more locations. Alternatively, in some other embodiments, the motor may be soldered, welded or secured to the expandable cage using a suitable adhesive material. One or more combinations of any of these securement means may also be used in accordance with the present disclosure to secure the motor to the expandable cage at one or more locations.

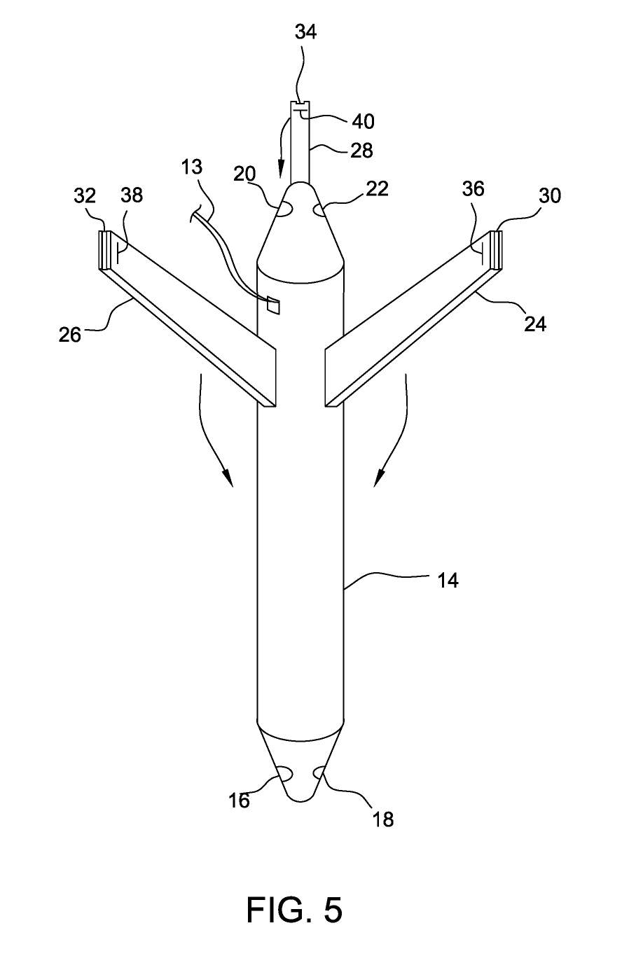

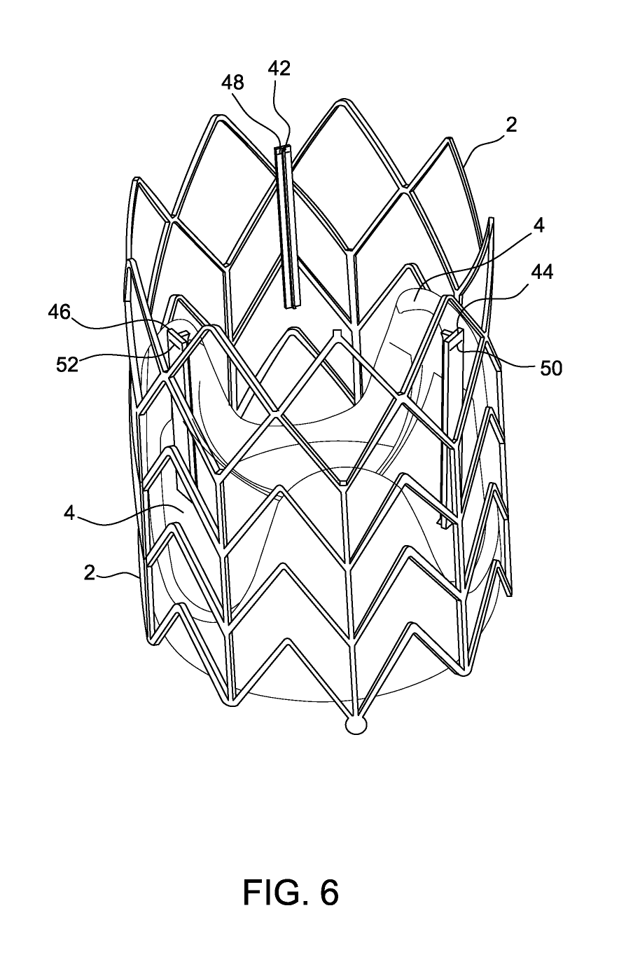

Referring now to FIG. 5, there is shown pump 14 having driveline 13, blood inlets 16 and 18 and blood outlets 20 and 22 for pumping blood through pump 14. Blood inlets 16 and 18 and blood outlets 20 and 22 may be shaped, sized and configured as desired to allow for blood to move therethrough. The inlets and outlets may be the same or different sizes, and may have any desired shape and size to allow the desired blood flow path. Pump 14 additionally includes fixation fins 24, 26, and 28 to allow for securement of pump 14 to an expandable frame (not shown in FIG. 5 but see FIG. 6). Fixation fins 24, 26, and 28 are desirably foldable or collapsible or otherwise configured to allow fixation fins 24, 26, and 28 to fold or collapse into pump 14 along the arrows indicated in FIG. 5 such that pump 14 may be easily deliverable and deployable through the use of a delivery catheter or delivery sheath as further described herein. In one suitable embodiment, fixation fins 24, 26, and 28 may be spring loaded to allow fixation fins 24, 26, and 28 to be collapsed for delivery and expand outward for securement upon release from a delivery device.

Referring again to FIG. 5, each fixation fins 24, 26, and 28 includes a recess 30, 32, and 34, respectively, sized and configured for cooperation with a rail system (not shown in FIG. 5 but see FIG. 6) installed on the expandable frame. The recess 30, 32, and 34 allow for securement of fixation fins 24, 26, and 28 to the expandable frame as well as for allowing a tactile signal to the operator deploying the pump into the expandable frame inside of the aortic root; that is, when recess 30, 32, and 34 are properly positioned and engage the rail system positioned on the expandable frame as described below, the operator can feel the connection and engagement, thus receiving a signal that the positioning is correct and engagement and securement may be continued. Alternatively, or in combination, one or more fixation fins 24, 26, and 28 may include a magnet (not shown in FIG. 5) that could contact and connect with a magnet on the expandable frame to produce a tactile signal that alignment is correct. Additionally, each of fixation fins 24, 26, and 28 may include a radiopaque marker 36, 38, and 40, respectively, thereon to further provide feedback of their location inside of the aortic root as the implantation and deployment procedure may be conducted using fluoroscopy or another visualization technique that provides a visualization of the radiopaque marker. Radiopaque markers 36, 38, and 40 on fixation fins 24, 26, and 28 may be aligned with corresponding radiopaque markers on the expandable frame (not shown in FIG. 5 but see FIG. 6). As such, an operator may be able to properly align fixation fins 24, 26, and 28 using a visual cue (i.e., radiopaque markers 36, 38, and 40) along with the tactile cues noted above to ensure proper placement and securement to the expandable frame.

As noted herein, the expandable frame desirably includes a receiving means that acts as a securement mechanism to secure the pump (or fixation fins or other means attached to the pump) to the expandable frame to secure the pump in a desired location during use. Referring now to FIG. 6, there is shown expandable frame 2 including prosthetic valve 4 and further including rails 42, 44, and 46 positioned on expandable frame 2 for engaging one or more fixation fins as described above to secure the pump to expandable frame 2. Each of rails 42, 44, and 46 includes a radiopaque marker 48, 50, and 52, respectively, to allow alignment of fixation fins 24, 26, and 28 with rails 42, 44, and 46 as described above.

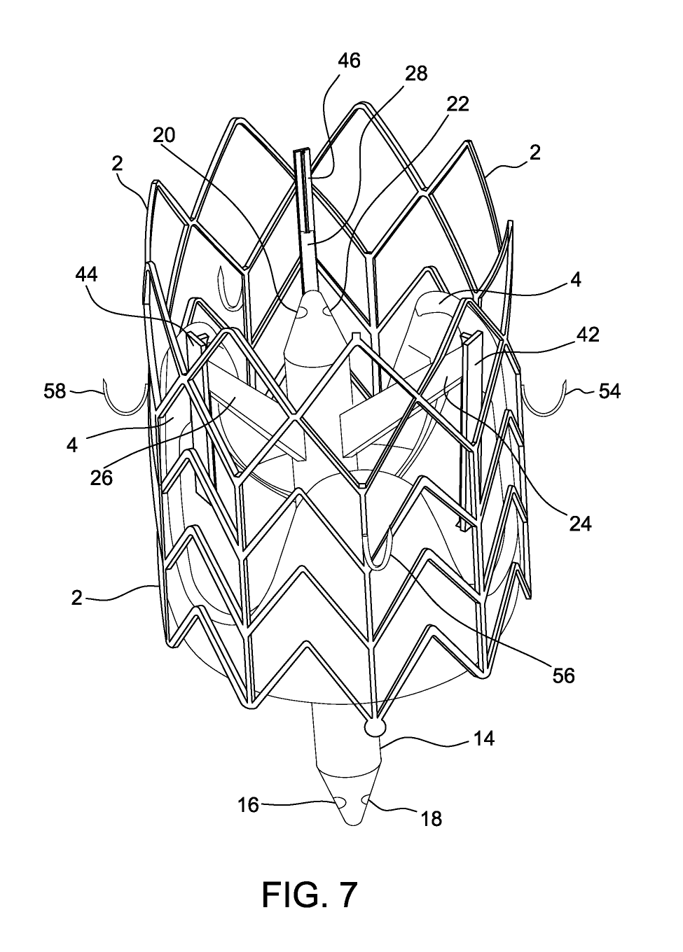

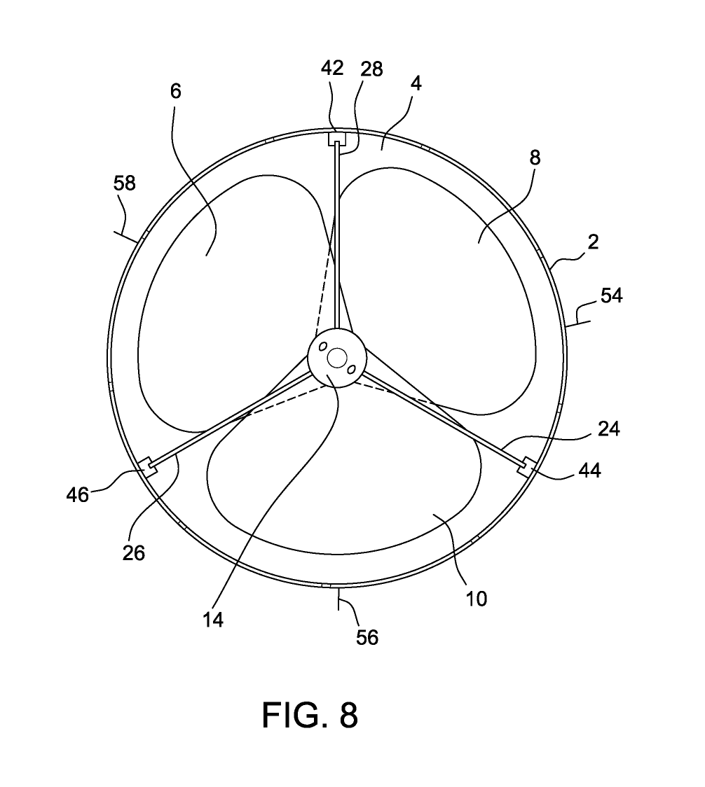

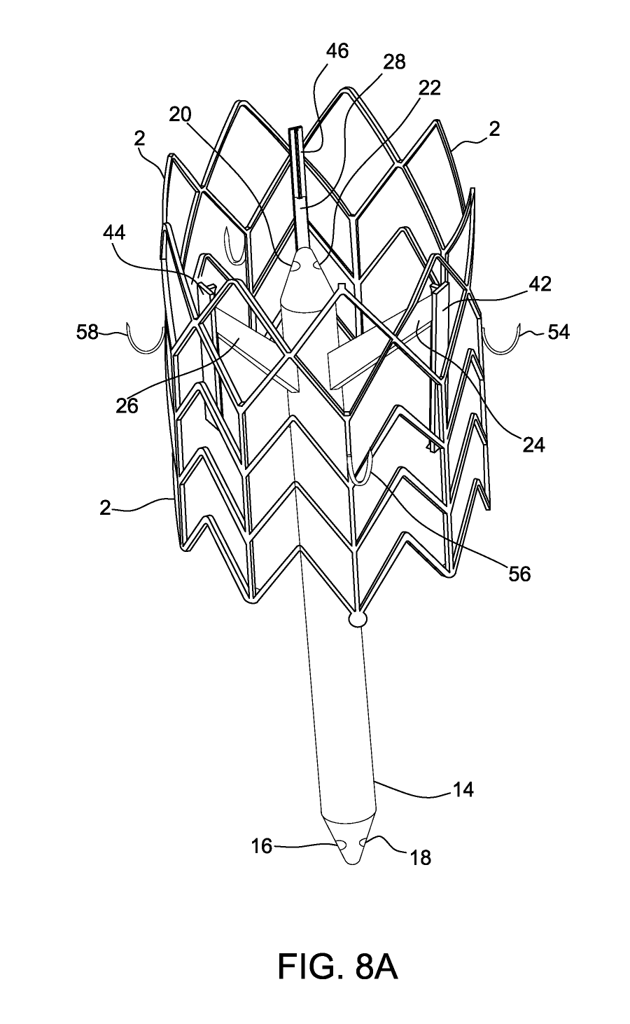

Referring now to FIG. 7, there is shown expandable frame 2 including prosthetic valve 4 wherein pump 14 having blood inlets 16 and 18 and blood outlets 20 and 22 is secured to expandable frame 2. Specifically, FIG. 7 shows fixation fins 24, 26, and 28 secured to rails 42, 44, and 46 respectively. Each of rails 42, 44, and 46 may include one or more stop points (not shown in FIG. 7) at which point each fixation fin will no longer move along the rail indicating to the operator that the fixation fin is properly located and fit on the rail and at an end point. Additionally, each rail and/or fixation fin may include one or more locking mechanisms (not shown in FIG. 7) to allow the fin to be locked to the rail system; such locking mechanisms may include a turn-style locking mechanism and the like. FIG. 7 additionally shows hooks 54, 56, and 58 on expandable frame 2. Hooks 54, 56, and 58 may be used to secure expandable frame 2 within the aortic root. Hooks 54, 56, and 58 allow expandable frame 2 to be properly positioned within the aortic root and be secured to the aortic root for an extended period of time. Alternatively, other securements means, such as barbs, may be used in place of, or in combination with, hooks 54, 56, and 58 for securement of expandable frame 2 within the aortic root. In some embodiments of the present disclosure, pump 14 and prosthetic valve 4 may be secured into expandable frame 2 such that they are coaxially aligned, generally along a major longitudinal axis of expandable frame 2 (See FIG. 8 below). In other embodiments, pump 14 and prosthetic valve 4 may not be coaxially aligned within expandable frame 2.

Referring now to FIG. 8, there is shown a top down view of an expandable frame 2 including a prosthetic valve 4 and pump 14 disposed therein wherein prosthetic valve 4 and pump 14 are coaxially aligned. Expandable frame 2 includes rails 42, 44, and 46, prosthetic valve 4 including valve leaflets 6, 8, and 10, pump 14 including fixation fins 24, 26, and 28, and hooks 54, 56, and 58. Valve leaflets 6, 8, and 10 (shown in closed conformation) wrap around and seal pump 14 as when the heart is resting as described above to allow a seal to form around pump 14 to keep blood from leaking back through prosthetic valve 4 upon the resting of the heart. Additionally, by aligning fixation fins 24, 26, and 28 coaxially with valve leaflets 6, 8, and 10, prosthetic valve 4 can operate as desired without interference from fixation fins 24, 26, and 28 or pump 14.

In one alternative embodiment of the present disclosure, a ventricular assist device as described herein may be implanted, deployed, and secured in the aortic root such that the native aortic valve, or a previously implanted prosthetic aortic valve, is not impeded or otherwise held open by the expandable frame and may continue to operate naturally; that is, in this embodiment, the ventricular assist device is implanted, deployed, and secured at a location in the aortic root above the native aortic valve such that the native aortic valve is not held in an open conformation and can freely open and close. In this embodiment, the pump may be secured into the expandable frame and the expandable frame positioned in the aortic root such that the pump extends out of the expandable frame such that it is positioned within the native aortic valve to assist in improving blood flow while allowing the native aortic valve to continue to function. The expandable frame may include a prosthetic valve disposed therein to further blood flow within the individual, or may be free of a prosthetic valve. In many embodiments of the present disclosure, the intravascular ventricular assist devices continue to provide a valuable function and assistance even if a prosthetic valve included therein has decreased or no performance.

Referring not to FIG. 8A, there is shown expandable frame 2 including rails 42, 44, and 46, pump 14 including blood inlets 16 and 18, blood outlets 20 and 22, and fixation fins 24, 26, and 28, and hooks 54, 56, and 58. In FIG. 8A, pump 14 is sized and configured to extend further out of expandable frame 2 such that pump 14 may reach across a native aortic valve (not shown in FIG. 8A) and into the left ventricle (not shown in FIG. 8A) to further blood flow within the individual. In this embodiment, a prosthetic valve (not shown in FIG. 8A) may optionally be used in combination with expandable frame 2 and pump 14 but is not generally required as pump 14 may interact with a functioning native aortic valve.

As noted above, the pump as described herein may be electrically or otherwise connected to a power supply and/or a controller and/or other peripherals using a driveline. In many embodiments, the power supply/controller may be in electrical communication (via hard wire or wirelessly) with another cardiac device, such as an implantable cardioverter defibrillator, or the like. In one specific alternative embodiment, the expandable frame may be wired to the power supply/controller and/or other peripherals such that electrical power is supplied to the pump an the interconnection of one or more of the fixation fins/rails; that is, power may be supplied to the pump through a connection of a fixation fin with a rail such that the connection provides a securement benefit and an electrical benefit. This embodiment may allow for the routing of the driveline to be completely separate of the prosthetic valve.

The intravascular ventricular assist devices of the present disclosure may be implanted/deployed using a number of suitable methods as described herein. The implanting and deployment of the devices may be done in a single step process wherein the intravascular ventricular assist device is implanted in a fully assembled state in a single step and subsequently deployed in the aortic root; that is, the intravascular ventricular assist device is preassembled outside of the body and implanted/deployed as a single unit in the aortic root. Alternatively, the intravascular ventricular assist device may be implanted in a two-step process where the expandable frame having the prosthetic valve disposed therein is first implanted and deployed followed by the implanting/securing of the pump into the expanded frame; that is, the expandable frame/prosthetic valve is first implanted and deployed and then the pump, which may be tethered to the expandable frame, is introduced into the expanded frame/prosthetic valve and secured to the expanded frame. This two-step process may allow for the delivery of a lower profile system (lower profile as the pump is introduced separately) that may be advantageous for some adults as well as pediatrics. The lower profile system may additionally potentially result in less trauma to the ascending aorta during implantation.

In one specific embodiment of the present disclosure wherein the intravascular ventricular assist device is fully assembled outside of the body, the operator gains access to the individual's ascending aorta such that a guidewire may be passed through the native aortic valve and into the left ventricle. Access to the ascending aorta can be obtained using any suitable procedure such as, for example a limited anterior thoracotomy, a limited sternotomy, through the axillary artery, through the transfemoral artery, a transapical procedure, and the like. After accessing the ascending aorta, the operator may optionally sew a graft onto the ascending aorta. The graft may have a length of, for example, from about 5 to about 15 millimeters and may act to protect the ascending aorta during the procedure. Also, the graft may allow for a larger diameter intravascular ventricular assist device to be passed therethrough with less trauma to the ascending aorta while improving the ability of the operator to close the aorta after the procedure is complete.

Once access to the ascending aorta is obtained and the optional graft applied, a guidewire is introduced through the native aortic valve and into the left ventricle. A suitable visualization technique, such as fluoroscopy, may be used to assist in tracking the guidewire to its desired destination, as well as throughout the entire procedure. After insertion of the guidewire, the assembled intravascular ventricular assist device is passed over the guidewire and positioned at the desired location in the aortic root. Again, a suitable visualization technique, such as fluoroscopy, may be used to position the intravascular ventricular assist device in the desired location. After positioning of the intravascular ventricular assist device is complete in the aortic root, the intravascular ventricular assist device is deployed (via the self-expanding frame or via balloon expansion) such that it contacts the aortic root. After deployment, the deployment device is removed over the guidewire and the guidewire is removed. Any driveline present on the pump will generally exit the individual at the same site where the guidewire was inserted. If a graft was sewn onto the ascending aorta, any driveline may be optionally sewn onto the graft for added stability. The driveline is then connected to the power supply/controller. In some embodiments, the controller will additionally be connected to an implantable cardioverter defibrillator or other cardiac device to allow for communication therebetween as discussed above.

In a two-step implanting procedure, the intravascular ventricular assist device is not fully assembled outside of the body but rather is fully assembled in the aortic root. Once the guidewire is in place, the expandable frame/prosthetic valve is first introduced over the guidewire and deployed (via the self-expanding frame or via balloon expansion) such that it contacts the aortic root. Next, the pump, which may be tethered or otherwise attached to the expandable frame/prosthetic valve to facilitate proper placement within the expandable frame, is inserted into the expanded frame and secured thereto as described herein. The tether or other attachment means acts as a guiding means to assist placement of the pump in the proper formation within the expanded frame. In one specific embodiment, the driveline for supplying electricity to the pump may be used to tether the pump to the expandable frame. After the pump is secured to the expandable frame, the deployment device is removed over the guidewire and the guidewire is removed. In some embodiments, a driveline that is used to power the pump may be used as a sole or partial tether to guide the pump into the desired location. The driveline present on pump will generally exit the individual at the same site where the guidewire was inserted. If a graft was sewn onto the ascending aorta, any electrical power wires may be optionally sewn onto the graft for added stability. The driveline is then connected to the power supply/controller. In some embodiments, the controller will additionally be connected to an implantable cardioverter defibrillator or other cardiac device to allow for communication therebetween. As noted herein, the pump may be secured to the expandable frame using a tethering system and/or an alternative securement system, such as a fixation fin/rail system as described above. If a fixation fin/rail system is utilized, the operator may again use fluoroscopy to align the fixation fins and rails using the radiopaque markers described herein.

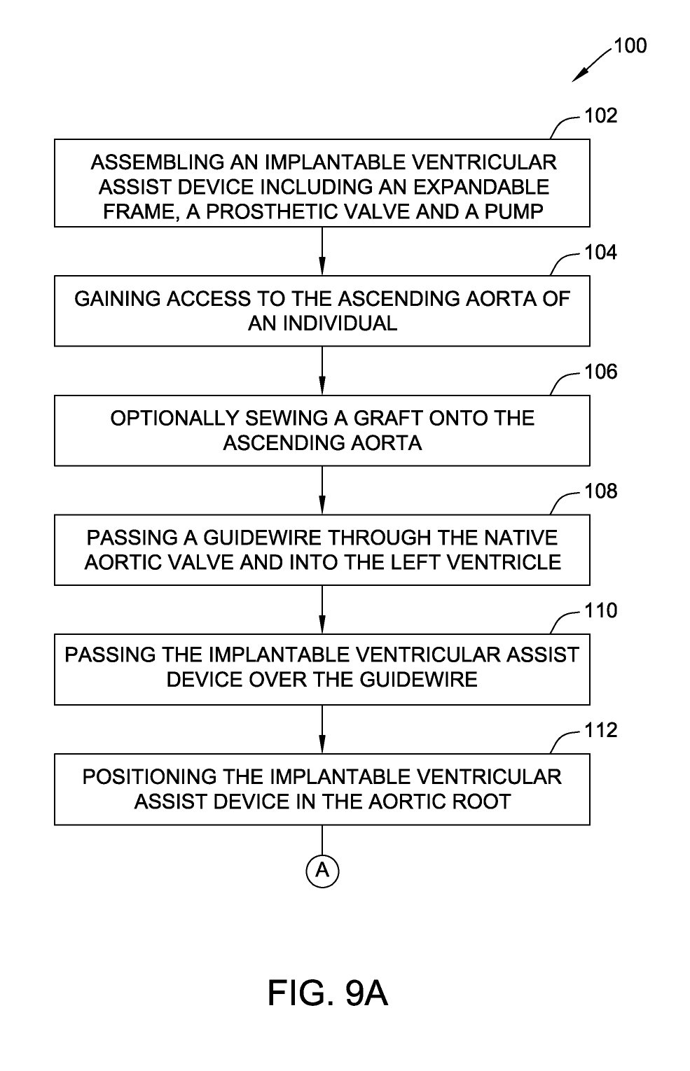

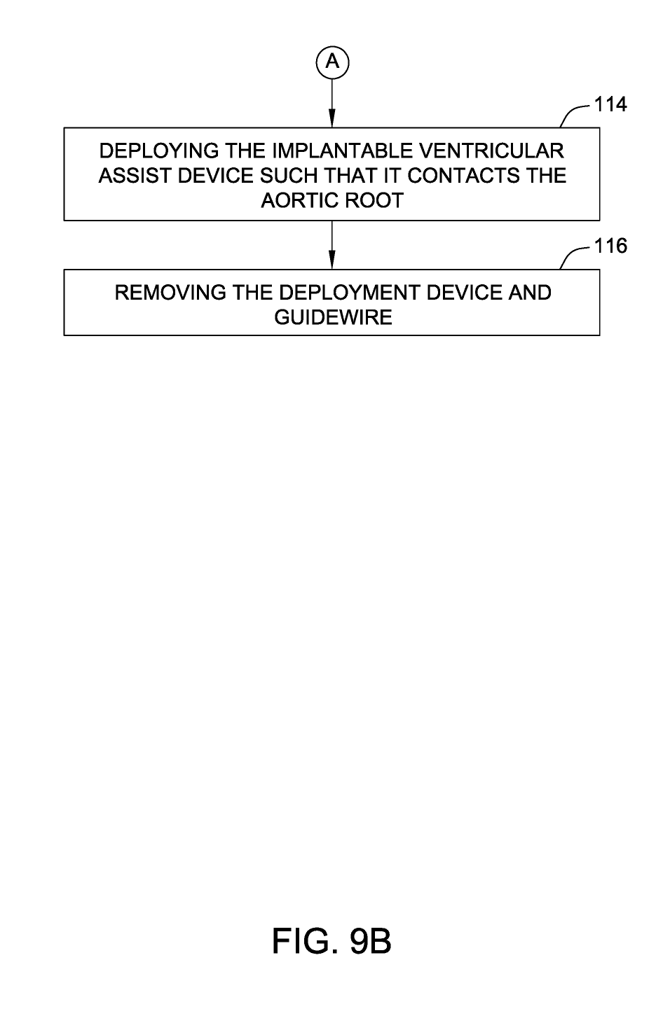

FIGS. 9A and 9B are flow charts of one embodiment of a method 100 for implanting an intravascular ventricular assist device. Method 100 comprises: (i) assembling 102 an intravascular ventricular assist device including an expandable frame, a prosthetic valve, and a pump; (ii) gaining 104 access to the ascending aorta of an individual; (iii) optionally sewing 106 a graft onto the ascending aorta; (iv) passing 108 a guidewire through the native aortic valve and into the left ventricle; (v) passing 110 the intravascular ventricular assist device over the guidewire; (vi) positioning 112 the intravascular ventricular assist device in the aortic root; (vii) deploying 114 the intravascular ventricular assist device such that it contacts the aortic root; and (viii) removing 116 the deployment device and the guidewire.

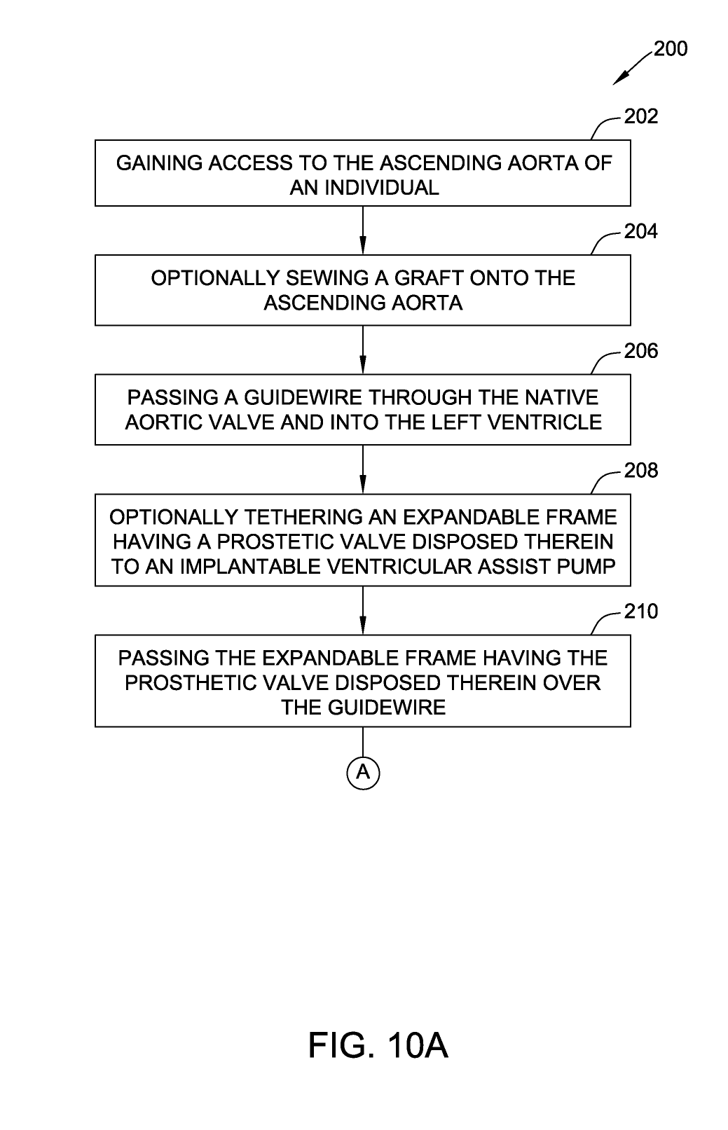

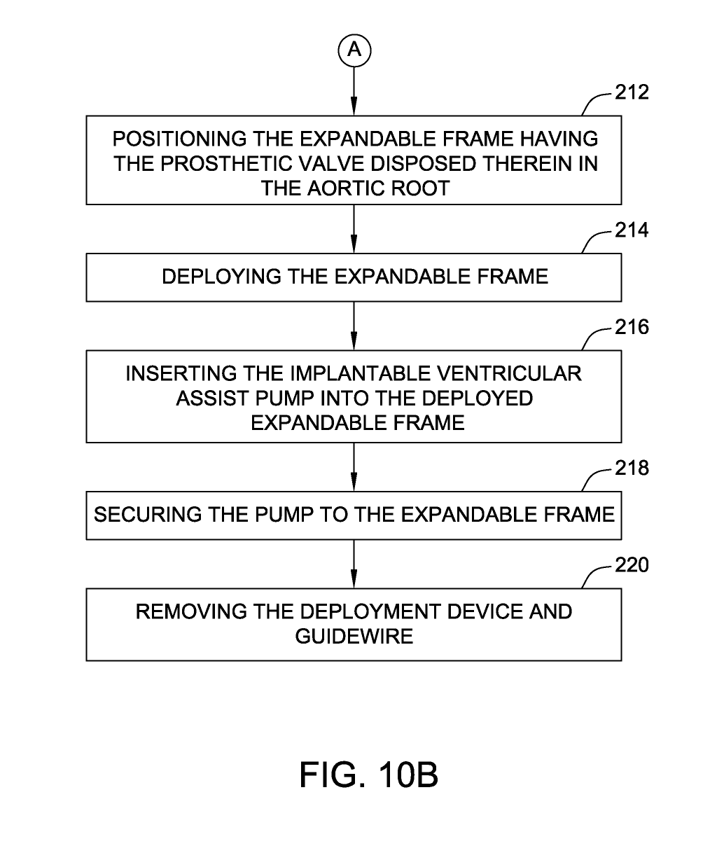

FIGS. 10A and 10B are flow charts of one embodiment of a method 200 for implanting an intravascular ventricular assist device. Method 200 comprises: (i) gaining 202 access to the ascending aorta of an individual; (ii) optionally sewing 204 a graft onto the ascending aorta; (iii) passing 206 a guidewire through the native aortic valve and into the left ventricle; (iv) optionally tethering 208 an expandable frame having a prosthetic valve disposed therein to an intravascular ventricular assist pump; (v) passing 210 the expandable frame having the prosthetic valve disposed therein over the guidewire; (vi) positioning 212 the expandable frame having the prosthetic valve disposed therein in the aortic root; (vii) deploying 214 the expandable frame; (viii) inserting 216 the intravascular ventricular assist pump into the deployed expandable frame; (ix) securing 218 the intravascular ventricular assist pump to the expandable frame; and (x) removing 220 the deployment device and guidewire.

Although a number embodiments of this disclosure have been described above with a certain degree of particularity, those skilled in the art could make numerous alterations to the disclosed embodiments without departing from the spirit or scope of the disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. An intravascular ventricular assist device comprising:
an expandable frame sized and configured for insertion into an aortic root, the expandable frame including a receiving means thereon; and
a pump disposed within the expandable frame and including attachment means configured to align with the receiving means disposed on the expandable frame to allow securement of the pump to the expandable frame.

2. The intravascular ventricular assist device of claim 1, further comprising:
a valve disposed in the expandable frame.

3. The intravascular ventricular assist device of claim 2, wherein the pump is axially aligned with the valve within the expandable frame.

4. The intravascular ventricular assist device of claim 3, wherein the pump and the valve are positioned along a major longitudinal axis of the expandable frame.

5. The intravascular ventricular assist device of claim 1, wherein the attachment means comprises one or more fixation fins sized and configured to cooperate with the receiving means to allow securement of the pump to the expandable frame.

6. The intravascular ventricular assist device of claim 5, wherein the attachment means includes three fixation fins each sized and configured to cooperate with three respective receiving means to allow securement of the pump to the expandable frame.

7. The intravascular ventricular assist device of claim 5, wherein the receiving means comprises a rail system comprising one or more receiving rails configured to cooperate with the attachment means.

8. The intravascular ventricular assist device of claim 7, wherein the one or more receiving rails comprise a locking mechanism configured to cooperate with a locking mechanism of a respective fixation fin.

9. The intravascular ventricular assist device of claim 1, wherein the expandable frame includes an attachment means for attaching the expandable frame to the aortic root.

10. The intravascular ventricular assist device of claim 9, wherein the attachment means for attaching the expandable frame to the aortic root includes one or more hooks or barbs.

11. The intravascular ventricular assist device of claim 1, wherein the expandable frame is self-expanding.

12. The intravascular ventricular assist device of claim 1, wherein the expandable frame is balloon expandable.

13. The intravascular ventricular assist device of claim 1, wherein the expandable frame comprises nitinol.

14. The intravascular ventricular assist device of claim 2, wherein the valve is selected from the group consisting of mechanical artificial valves and tissue artificial valves.

15. The intravascular ventricular assist device of claim 1, wherein the pump further comprises:
one or more drivelines to allow for electrical communication with a peripheral.

16. The intravascular ventricular assist device of claim 1, wherein the pump further comprises:
a means to allow transcutaneous energy transfer.

17. The intravascular ventricular assist device of claim 1, further comprising:
a means for electrically connecting the pump to an implantable cardioverter defibrillator.

18. The intravascular ventricular assist device of claim 1, further comprising a tether connecting the expandable frame and the pump.

19. A process for implanting an intravascular ventricular assist device, the process comprising:
inserting a guidewire through a native aortic valve;
delivering over the guidewire an expandable frame comprising a valve disposed therein into an aortic root
deploying the expandable frame comprising the valve; and
delivering a pump into the deployed expandable frame.

20. A process for implanting an intravascular ventricular assist device, the process comprising:
inserting a guidewire through a native aortic valve;
delivering over the guidewire an intravascular ventricular assist device, wherein the intravascular ventricular assist device comprises:
an expandable frame sized and configured for insertion into an aortic root;
a valve disposed in the expandable frame;
a pump disposed within the expandable frame and including attachment means configured to align with receiving means disposed on the expandable frame to allow securement of the pump to the expandable frame.
